(19) United States
(12) Patent Application Publication
(10) Pub. No.: US 2014/0113840 A1
(43) Pub. Date: Apr. 24, 2014

(54) ANALYTE DETECTION SYSTEM WITH CLEANING PHASE AND RENEWABLE LIQUID SENSING MATERIAL AND METHODS THEREFORE

(71) Applicant: EMPIRE TECHNOLOGY DEVELOPMENT LLC, Wilmington (DE)

(72) Inventors: Mordehai Margalit, Zichron Yaaqov (IL); Gil Perlberg, Zichron Yaaqov (IL)

(73) Assignee: EMPIRE TECHNOLOGY DEVELOPMENT LLC, Wilmington, DE (US)

(21) Appl. No.: 13/825,491

(22) PCT Filed: Oct. 19, 2012
(86) PCT No.: PCT/US12/60938

§ 371 Date: Mar. 21, 2013

(51) Int. Cl. G01N 33/543; C12Q 3/00

(52) U.S. Cl. CPC G01N 33/54373; C12Q 3/00
USPC 506/39; 422/81; 422/69; 436/501; 436/43; 435/3; 435/286.5

(57) ABSTRACT

Methods and systems for detecting analytes using a sensor element having a cleaning cycle and renewable liquid material with an affinity for the analytes are described. An analyte detection system may include a controller in communication with a sensor element and a liquid dispensing assembly. The liquid dispensing assembly, such as an inkjet dispensing device, may deposit the liquid material on or adjacent to the sensor element. The controller may be operative to monitor at least one property associated with the liquid material deposited on the sensor element. In addition, the controller may be configured to generate at least one control signal based on the at least one property. The liquid dispensing assembly may be configured to deposit the at least one liquid material on the sensor element based on the at least one control signal.

### BACKGROUND

Various methods have been developed to detect and measure chemical, gas, and biological analytes. Accurate methods are important because the ability to repeatedly and precisely detect analytes is a critical step in many processes, such as environmental testing, immunoassays, and the production of high purity compounds. One method involves exposing samples to materials having a high affinity for a substance of interest potentially in the sample. The high-affinity material will interact with the substance of interest, generating a reaction having one or more detectable properties. Illustrative properties include conductance, transparency, color, and temperature. The properties and/or changes in the properties are used by the sensor to detect and measure the substance of interest.

Typical high-affinity sensor systems include a dispensing apparatus configured to deposit the high-affinity material such that it contacts a sample in midair or on the surface of a sensor. In certain high-affinity sensor systems, the dispensing apparatus is an inkjet device that releases the high-affinity material as droplets of relatively uniform size and shape. One deficiency of high-affinity sensors is the degradation of the high-affinity materials over time. Another deficiency is the inability to efficiently measure a substance of interest with varying levels of sensitivity, particularly using a single sensor apparatus. Although high-affinity sensors are an important measurement tool, these deficiencies operate to limit the effectiveness of such sensors for detecting and measuring analytes.

### SUMMARY

This disclosure is not limited to the particular systems, devices and methods described, as these may vary. The terminology used in the description is for the purpose of describing the particular versions or embodiments only, and is not intended to limit the scope.

As used in this document, the singular forms “a,” “an,” and “the” include plural references unless the context clearly dictates otherwise. Unless defined otherwise, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention. As used in this document, the term “comprising” means “including, but not limited to.”

In an embodiment, a system for measuring at least one substance using at least one liquid material may comprise a sensor element, which comprises a controller, and a liquid dispensing assembly in communication with the controller. The controller may be operative to monitor at least one property associated with the at least one liquid material deposited on the sensor element. In addition, the controller may be configured to generate at least one control signal based on the at least one property. The liquid dispensing assembly may be configured to deposit the at least one liquid material on the sensor element based on the at least one control signal.

In an embodiment, a method of preparing a sensor element for measuring at least one substance using at least one liquid material may comprise providing a sensor element configured to receive the at least one liquid material. A controller may be configured to monitor at least one property associated with the at least one liquid material deposited on the sensor element and to generate at least one control signal based on the at least one property. A liquid dispensing assembly may be arranged to be in communication with the controller. In addition, the liquid dispensing assembly may be configured to deposit the at least one liquid material on the sensor element responsive to receiving the at least one control signal.

In an embodiment, a method of measuring at least one substance in a target material using a sensor element configured to measure the at least one substance using at least one liquid material may comprise arranging a sensor element to receive the at least one liquid material and arranging a liquid dispensing assembly to deposit the at least one liquid material on the sensor element. The sensor element may comprise a controller operative to monitor at least one property associated with the at least one liquid material deposited on the sensor element and to generate at least one control signal based on the at least one property. The liquid dispensing assembly may be configured to be in operable communication with the controller and to deposit the at least one liquid material on the sensor element based on the at least one control. The at least one liquid material may be exposed to the target material such that the sensor element measures for the at least one substance in the target material.

In an embodiment, a system for measuring at least one substance using at least one liquid material may comprise a sensor element, a cleaning element, and a liquid dispensing assembly. The sensor element may comprise a controller operative to monitor at least one property associated with the at least one liquid material deposited on the sensor element. The controller may be configured to generate at least one control signal based on the at least one property. The cleaning element may be operative to clean the sensor element by removing the at least one liquid material deposited on the sensor element based on the at least one control signal. The liquid dispensing assembly may be in communication with the controller and may be configured to deposit the at least one liquid material on the sensor element responsive to the sensor element being cleaned.

### BRIEF DESCRIPTION OF THE FIGURES

FIG. 1 depicts an illustrative analyte detection system for measuring a substance using a liquid material according to some embodiments.

FIG. 2 depicts another illustrative analyte detection system for measuring a substance using a liquid material according to some embodiments.

FIG. 3 depicts a flow diagram for an illustrative method of preparing a sensor element according to some embodiments.

FIG. 4 depicts a flow diagram for an illustrative method of measuring a substance using a sensor element configured according to some embodiments.

FIG. 5 depicts an illustrative computing device that may be used to contain or implement program instructions for controlling aspects of an analyte detection system according to some embodiments.

### DETAILED DESCRIPTION

The present disclosure is directed to an analyte detection system comprising a sensor element, a liquid dispensing assembly, and a controller configured to control the liquid dispensing assembly. In an embodiment, the controller may monitor at least one property of a liquid material deposited on or around the sensor element by the liquid dispensing assembly. The liquid material may be deposited such that it contacts a target material being examined for a substance or substances of interest. According to some embodiments, the at least one property may indicate an effectiveness of the liquid material at measuring the substance or substances of interest. The controller may communicate control signals to the liquid dispensing assembly to control the release of the liquid material to, among other things, manage the effectiveness of the liquid material. In another embodiment, the sensor element may be cleaned to remove degraded liquid material. In a further embodiment, the liquid dispensing assembly may be configured to deposit a plurality of liquid materials. For instance, the liquid dispensing assembly may comprise a plurality of reservoirs holding different liquid materials or different forms of the same liquid material (e.g., liquid materials having a different sensitivity to a substance of interest).

The following terms shall have, for the purposes of this application, the respective meanings set forth below.

A “sensor element” refers to an element configured to detect the presence of and/or measure a substance or substances. Typically, a sensor element operates by detecting a change in a property resulting from the interaction of a target material and a liquid material (defined below) having an affinity for a substance of interest in the target material. For example, a sensor element may be configured to measure capacitance, mass, fluorescence, or optical properties. A liquid material may be deposited on the sensor element such that it contacts a target material being measured for the presence of a substance of interest. When the liquid material contacts the substance of interest, a detectable change in a property occurs, such as an increase in capacitance, which may be detected by the sensor element. The sensor element may further operate to measure an amount of the substance of interest based on the change in the property (e.g., the greater the change in the property, the larger the amount of the substance, etc.).

A “liquid material,” as used in reference to a sensor element, refers to a liquid, liquid mixture, liquid-gas mixture, or liquid solution that interacts with a substance of interest in a manner that is detectable and/or measurable by the sensor element. For example, the liquid material may have a high affinity for a substance of interest such that the liquid material chemically or physically reacts with a substance of interest. A sensor element may be configured to detect one or more properties associated with the chemical or physical reaction.

A “substance,” as used in reference to a sensor element, refers to matter that may be measured by a sensor element using, among other things, a liquid material. A substance may be in various forms, such as liquid, gas, solid, intermediate states, and combinations thereof. As used herein, a “substance of interest” is a substance or substances that are the focus of detection and/or measurement by a sensor element.

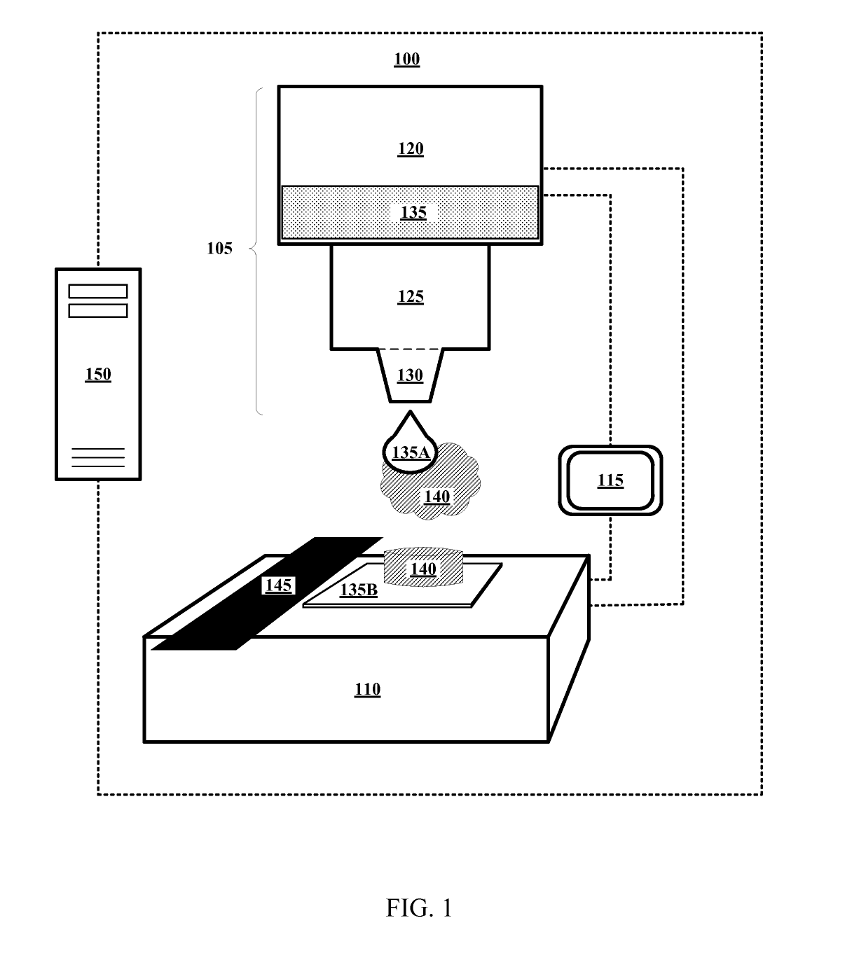

FIG. 1 depicts an illustrative analyte detection system according to some embodiments. As shown in FIG. 1, an analyte detection system 100 may comprise a sensor element 110 arranged to receive a liquid material 135 deposited by a liquid dispensing assembly 105. The liquid material 135A comprises liquid material that has been released by the liquid dispensing assembly 105 and is in the process of being deposited onto the sensor element 110. The liquid material 135B comprises liquid material that has already been deposited on the sensor element 110. In an embodiment, the liquid material 135B may bind to the sensor element 110 and/or a target material 140 contacted by the liquid material.

In an embodiment, the sensor element 110 may comprise at least one of the following: an affinity sensor, an immunosensor, a metabolism sensor, a catalytic sensor, an electrochemical sensor, a fluorescent sensor, a surface acoustic wave (SAW) sensor, an attenuated total reflection (ATR) sensor, a piezoelectric sensor, a piezoelectric quartz crystal sensor, a thickness shear mode sensor, a flexural plate wave sensor, an optical absorption sensor, a Fourier-transform infrared (FTIR) optical sensor, a Raman optical sensor, an electrical properties sensor, a magnetic properties sensor, a conductance sensor, a capacitance sensor, a magnetic field orientation sensor, and a bulk acoustic wave resonator sensor.

In an embodiment, the liquid material 135 may comprise at least one of the following: antibodies, antigens, enzymes, polymers, silicone polymers, carbowax polymers, cyclodextrin polymers, and fluorescent indicators.

In an embodiment, the liquid material 135 may comprise a high-affinity material configured to detectably react with a substance or substances of interest. For example, the liquid material 135 may measurably react with a target material 140 such that the sensor element 110 detects the presence of and/or measures an amount of a substance of interest associated with the target material. In an embodiment, the analyte detection system 100 may be configured to detect the presence of a substance or substances of interest (e.g., a yes/no analysis). In another embodiment, the analyte detection system 100 may be configured to measure an amount of a substance or substance of interest. The measurements may be based on the detectable reaction of the liquid material 135 with a substance of interest that is measured by the sensor element 110.

As depicted in FIG. 1, the liquid material 135 may contact the target material 140 in midair in the space between the liquid dispensing assembly 105 and the sensor element 110 and/or the liquid material may contact the target material on a surface of the sensor element. Accordingly, the liquid material 135 may interact with the target material 140 in flight from the liquid dispensing assembly 105 to the sensor element 110 and/or the liquid material may interact with the target material on the surface of the sensor element.

The liquid dispensing assembly 105 may comprise a reservoir 120, an actuator 125 and a nozzle 130. The reservoir 120 may be configured to hold one or more liquid materials 135. The actuator 125 may be in fluid communication with the reservoir 120 and may be configured to discharge the liquid material 135 from the reservoir and into the nozzle 130. The nozzle 130 may be arranged to deposit droplets of the liquid material 135 on the sensor element 110. In an embodiment, the liquid dispensing assembly 105 comprises an inkjet printer assembly.

In an embodiment, the actuator 125 comprises one of the following: a piezoelectric actuator, an acoustic actuator, piezo-acoustic actuator, a thermal actuator, and an electrostatic actuator.

The distance between the liquid dispensing assembly 105 and the sensor element 110 may be configured based on one or more liquid dispensing characteristics, including, without limitation, the characteristics associated with the sensor element, target material, substance of interest, and liquid material. Such characteristics may include typical droplet size, speed, volatility, and target speed for landing on sensor. Sensor elements may have limitations in size, for example, the sensor may require sufficient clearance for guiding illumination or irradiation light to a sample. In an embodiment, the distance between the nozzle 130 and the sensor element 110 may be about 3 mm to about 15 mm. In another embodiment, the distance between the nozzle 130 and the sensor element 110 may be about 5 mm to about 10 mm. In a further embodiment, the distance between the nozzle 130 and the sensor element 110 may be about 5 mm to about 9 mm. In a still further embodiment, the distance between the nozzle 130 and the sensor element 110 may be less than about 10 mm.

Additional liquid dispensing characteristics include, but are not limited to, droplet volume, size and speed. The liquid dispensing assembly 105 may be configured to repeatedly dispense the liquid material 135A in droplets having substantially uniform characteristics, for example, as specified by an operator of the analyte detection system 100. In an embodiment, droplet volume may be from about 10 picoliters to about 40 picoliters. In another embodiment, droplet volume may be from about 20 picoliters to about 30 picoliters. In a further embodiment, droplet volume may be from about 5 picoliters to about 50 picoliters. Specific examples of droplet volumes include about 10 picoliters, about 20 picoliters, about 30 picoliters, about 40 picoliters, and ranges between any two of these values (including endpoints).

In an embodiment, droplet radius may be about 10 μm to about 200 μm. In another embodiment, droplet radius may be about 10 μm to about 30 μm. In a further embodiment, droplet radius may be about 50 μm to about 20 μm. Specific examples of droplet sizes include about 10 μm, about 20 μm, about 30 μm, about 40 μm, about 50 μm, about 75 μm, about 100 μm, about 125 μm, about 150 μm, about 175 μm, and about 200 μm, and ranges between any two of these values (including endpoints). In an embodiment, droplet speed may be about 0.75 m/s to about 1.5 m/s. In another embodiment, droplet speed may be about 1 m/s to about 1.5 m/s. In a further embodiment, droplet speed may be about 0.5 m/s to about 2.0 m/s. Specific examples of droplet speeds include about 0.75 m/s, about 1 m/s, about 1.25 m/s, about 1.5 m/s, about 1.75 m/s, about 2 m/s, and ranges between any two of these values (including endpoints).

Although one liquid dispensing assembly 105, one controller 115 and one sensor element 110 are depicted in FIG. 1, embodiments are not limited to only one of each of these components, as the analyte detection system 100 may comprise multiple liquid dispensing assemblies, controllers and sensor elements. For example, the analyte detection system 100 may comprise four sensor elements 110 and two liquid dispensing assemblies 105. Furthermore, each liquid dispensing assembly 105 may be associated with two sensor elements 110 and two controllers 115, one for each sensor element. In this example, the liquid dispensing assemblies 105 may be associated with a motor or other device configured to move the liquid dispensing assemblies in position to deposit the liquid material 135 on or adjacent to each sensor element 110, as required. As such, the liquid dispensing assembly 105 may operate to deposit multiple liquid materials 135 and/or multiple concentrations of liquid materials without having to stop an analysis and change the liquid material in the liquid dispensing assembly.

In addition, the liquid dispensing assembly 105 is not limited to one reservoir 120, one actuator 125, and one nozzle 130, as the liquid dispensing assembly may comprise two or more of each component. In this manner, the liquid dispensing assembly 105 may operate to deposit multiple liquid materials 135 and/or multiple concentrations of liquid materials without having to stop an analysis and change the liquid material in the liquid dispensing assembly. For example, a liquid dispensing assembly 105 may comprise three reservoirs 120, each in fluid communication with an actuator 125 and a nozzle 130 corresponding to each actuator.

The liquid dispensing assembly 105 may be in communication with a controller 115 operative to monitor at least one property associated with the liquid material 135. The sensor element 110 may be configured to generate a control signal based on the at least one property. According to some embodiments, the controller 115 may comprise an element for measuring the at least one property and/or may receive measurement information from an external element that measures for the at least one property that is in communication with the controller. The control signal may be transmitted to the liquid dispensing assembly 105, the sensor element 110, and/or a cleaning element 145.

In an embodiment, the at least one property monitored by the controller 115 may indicate an effectiveness of the liquid material 135B at measurably reacting with a substance or substances of interest. For example, the controller 115 may be operative to measure a temperature of the liquid material 135B. As the temperature of the liquid material 135B decreases, the effectiveness of the liquid material also decreases. In another example, the controller 115 may comprise a timer configured to measure how long the liquid material 135 has been deposited on the sensor element 110 (or other relevant surface) or has been held in the reservoir 120. The controller 115 may determine that the effectiveness of the liquid material 135 is below a threshold level after the liquid material has been deposited on the sensor element 110 and/or has been held in the reservoir 120 for a predetermined time.

In an embodiment, the controller 115 may be in communication with the sensor element 110. For example, the controller 115 may be configured to receive measurement data from the sensor element 110 that may be used to measure at least one property of the liquid material.

In an embodiment, the at least one property may comprise at least one of the following: time, temperature, mass, conductance, transparency, color, fluorescence, refractive index, dielectric coefficient, magnetic coefficient, nonlinear response to stimuli, degradation characteristics, and substance absorption.

The control signal may operate to invoke the liquid dispensing assembly 105 to release more of the liquid material 135 and/or to stop releasing the liquid material, depending on the effectiveness of the liquid material as detected by the controller 115 and/or other analysis conditions (e.g., the analysis is complete). In this manner, the analyte detection system 100 may operate to control the flow of liquid material 135 based on, among other things, the effectiveness of the liquid material. For example, the liquid dispensing assembly 105 may renew the liquid material 135B on the sensor element 110 when at least one property indicates that the liquid material is inadequate to accurately measure the substance of interest. For example, the liquid material is inadequate if there is not enough of the liquid material 135B, as indicated by a mass of the liquid material, and/or the reactive property detectable by the sensor element 110 has degraded.

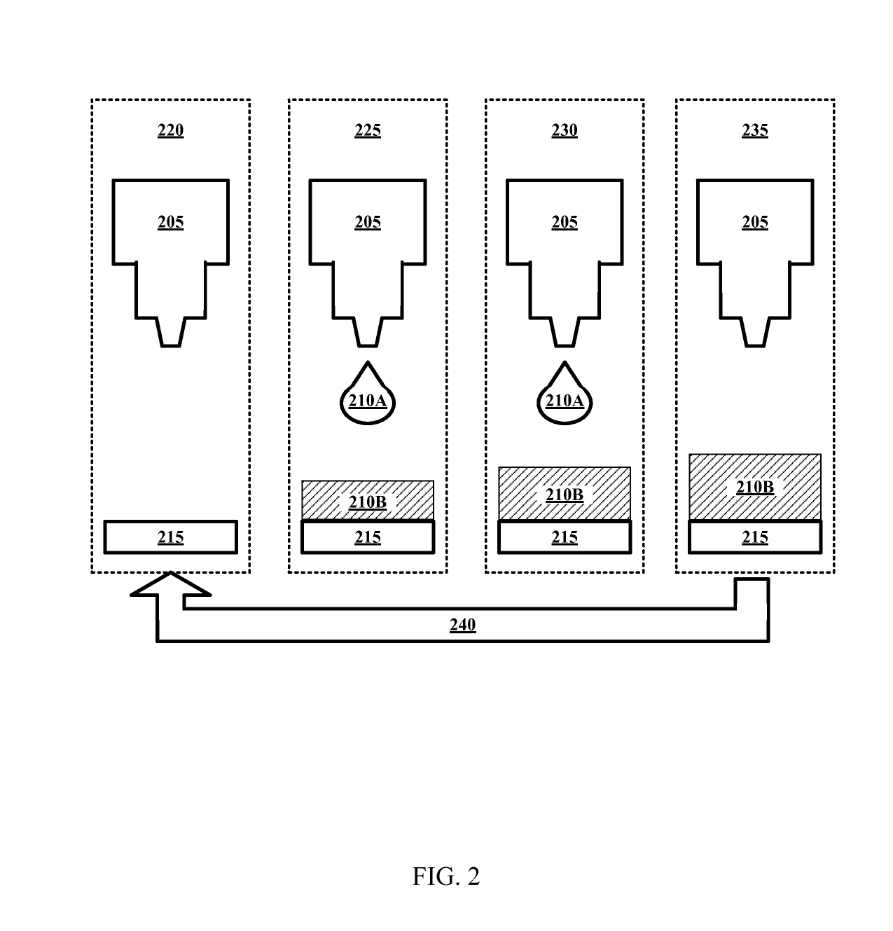

The sensor element 110 may be associated with a cleaning element 145 configured to clean (or “scrub”) the liquid material 135B from the sensor element, described in more detail with respect to FIG. 2, below. In an embodiment, the cleaning element 145 may comprise at least one of the following: an ultrasonic element, a thermal element, an electric element, an optical element, and a physical element. Some embodiments provide that the cleaning element 145 may operate to clean the sensor element 110 when the liquid material 135B becomes sufficiently degraded, as indicated by the controller 115 and/or the sensor element 110. The cleaning element 145 may also operate according to some embodiments to clean the sensor element 110 before a different liquid material 135 is deposited on the sensor element.

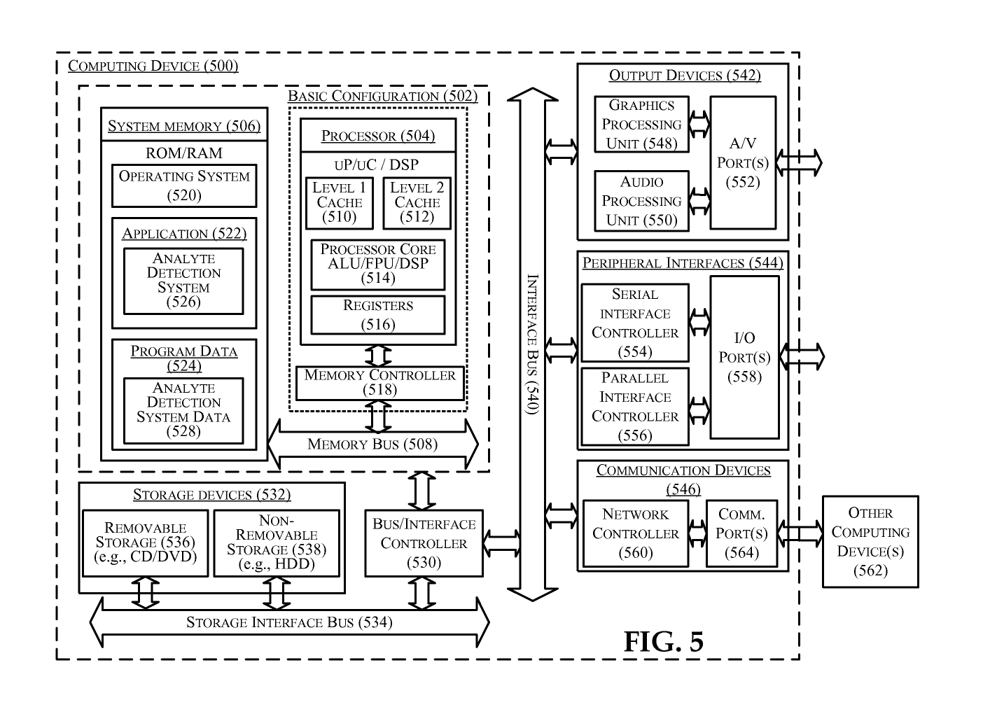

In an embodiment, the analyte measurement system 100 may comprise one or computing devices 150 and/or one or more processors and system memory (not shown), such as the computing device 500, processor 504 and system memory 506 depicted in FIG. 5 below. The computing device 150 may be associated with software configured to manage and/or control the analyte detection system 105, associated data, and components thereof as described herein. For example, a software application operating on the computing device 150 may receive information obtained by the controller 115 relating to at least one property of the liquid material 135. The software application may feed the information into a process configured to determine whether the liquid material 135 has degraded. If the software application determines that the liquid material 135 has degraded, the software application may operate to transmit a signal to the controller 115. The controller 115 may transmit a control signal to the liquid dispensing assembly 105 in response to the signal transmitted by the software application to dispense more liquid material 135.

In an embodiment, the analyte measurement system 100 may operate to perform one or more tests in series. The tests may comprise different measurement attributes, such as measuring both electrical and optical properties. In another embodiment, the tests may comprise a certain sequence of tests and/or activations. For example, a sample may be irradiated by a UV light and then its electrical characteristics measured over time.

FIG. 2 depicts another illustrative system for measuring a substance using a liquid material according to some embodiments. As shown in FIG. 2, a liquid dispensing assembly 205 may be configured to dispense a liquid material 210A, 210B on a sensor element 215. The liquid material 210A comprises liquid material that has been released by the liquid dispensing assembly 205 and is in the process of being deposited onto the sensor element 215. The liquid material 210B comprises liquid material that has already been deposited on the sensor element 215.

The sensor element 215 may progress through one or more phases, such as the four phases 220, 225, 230, and 235 depicted in FIG. 2. In a clean phase 220, no liquid material 210B is on the sensor element 215. In the material deposition phases 225 and 230, the liquid material 210A is released by the liquid dispensing assembly 205 and is deposited on the sensor element 215, for instance, until an adequate amount of liquid material 210B is present on the sensor element. An adequate amount of liquid material may depend on various factors, including, without limitation, the sensor element, liquid material, target material, substance of interest, and combinations thereof.

In an embodiment, the sensor element 215 and/or the controller (not shown) may be configured to measure for an adequate amount of deposited liquid material 210B. For instance, the adequate amount of liquid material 210B may be based on the mass of deposited liquid material. An illustrative and non-restrictive example provides for a SAW sensor element monitoring for resonance frequency, which is dependent upon the mass of the deposited liquid material 210B. In this manner, the resonance frequency may be used to control the amount of deposited liquid material 210B with high accuracy.

In the measurement phase 235, an adequate amount of liquid material 210B has been deposited on the sensor element 215, and the sensor element operates to measure for the substance of interest. The cleaning phase 240 may operate to clean the sensor element 215 and return the sensor element to the clean state 220. During the cleaning phase 240, the liquid material 210B may be removed using various methods. In an embodiment, the cleaning element may comprise at least one of the following: an ultrasonic element, a thermal element, an electric element, an optical element, and a physical element. For example, a heating element or an electric element may operate to burn off the liquid material 210B. In another example, an ultrasonic element may use sound waves to break up and dissipate the liquid material 210B. In a further example, a physical element may operate to scrape, push, scrub, spray, blow, or otherwise physically remove the liquid material 210B from the sensor element 215.

The four phases 220, 225, 230, 235 depicted in FIG. 2 are non-restrictive and are for illustrative purposes only. Some embodiments provide for more or fewer phases in one or more different sequences than depicted in FIG. 2. For example, measurement of a particular analyte may comprise a material deposition phase followed by a measurement phase followed by another material deposition phase. In this example, the pattern of material deposition phases and measurement phases may be repeated as necessary. In another example, there may be five deposition phases and one measurement phase that is followed by a cleaning phase. According to some embodiments, a measurement decision may be generated based on the cumulative results of multiple measurement phases, including fewer than all measurement phases (e.g., removing measurement phases with results that fall outside of an acceptable range).

Some embodiments provide for multiple cleaning elements that may be used in combination. For example, a heating element may operate to dry the liquid material 210B on the sensor element 215, and a physical element may then operate to scrape and/or push the dried liquid material off of the sensor element. In another example, an ultrasonic device may break up the liquid material 210B, while a physical element may spray a liquid on the sensor element 215 that pushes the liquid material off of the sensor element. Embodiments are not limited to the aforementioned examples, as any combination of cleaning elements capable of operating according to at least some of the described embodiments is contemplated herein.

The cleaning phase 240 may be initiated for various purposes. For example, the cleaning phase 240 may be initiated responsive to degradation of the liquid material 210B, a predetermined amount of time, environmental conditions, detection of an analyte, or a combination thereof.

The cleaning phase 240 may also be invoked to change the type of liquid materials 210A, 210B, for instance, to detect a different substance of interest or to measure the substance of interest at a different sensitivity level. In an embodiment, an initial measurement phase (e.g., including phases such as 220, 225, 230, and 235) may involve a liquid material 210A, 210B having a high sensitivity to the substance of interest to detect minute amounts of the substance of interest. A second measurement phase may be initiated after the sensor element 215 has been cleaned to measure the analyte using a liquid material 210A, 210B having a lower sensitivity. Measuring the substance of interest using liquid material 210A, 210B having varying levels of sensitivity may provide various benefits, such as preventing false positives, increased accuracy as high sensitivity materials may saturate more easily than low sensitivity materials, and more precise measurement of certain substances of interest, including variations and strands of a substance of interest.

In another instance, different substances of interest may be measured in the same or different target materials using the same sensor without having to complete a costly and time consuming transfer and process or switching sensor elements. An initial measurement phase may involve a liquid material 210A, 210B having an affinity for a particular substance of interest. A second measurement phase may be initiated after the sensor element 215 has been cleaned to measure another analyte using a different liquid material 210A, 210B having an affinity for a different substance of interest. In this manner, multiple substances may be measured for the same target material using the same sensor element 215.

In an embodiment, the sensor element 215 may be closed to the environment in the clean state 220 and during the cleaning phase 240. For example, some embodiments provide for fluid delivery (e.g., target material) to the surface of the sensor element 215. The sensor element 215 may be closed to the environment by blocking the fluid delivery. After the liquid material 210B has been deposited (e.g., phases 225 and 230), the sensor element 215 may be calibrated and the fluid delivery activated.

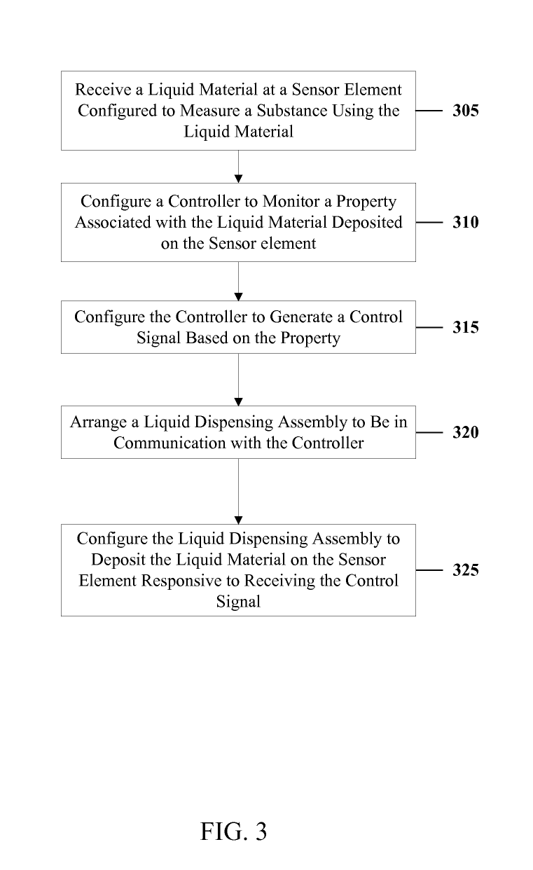

FIG. 3 depicts a flow diagram for an illustrative method of preparing a sensor element according to some embodiments. A liquid material may be received 305 at a sensor element configured to measure a substance using the liquid material. For example, a sensor element may measure a reaction of the liquid material with the substance. In an embodiment, the liquid material may be deposited on the sensor element up to a predetermined threshold. The liquid material may be exposed to a target material being measured for the presence of the substance delivered, for example, to the sensor element through a fluid delivery system. In another embodiment, the target material may be a gas delivered to an area surrounding the sensor element such that the substance in the target material may contact and react with the liquid material.

Certain types of sensor elements may be prepared according to different methods. For instance, in a SAW analyte detection system, chemically selective thin film coatings may be applied to a substrate that operates to shift the frequency of a SAW sensor. Adsorption of chemicals into the thin films further shifts the propagation velocity, which causes a change in the electrical output. Analytes may be detected by choosing coatings that selectively adsorb the analytes, such as explosive vapors. For example, nitroaromatic sensing SAW devices may be prepared with silicone polymers, carbowax polymers, and/or cyclodextrin polymers.

A controller may be configured to monitor 310 a property associated with the liquid material deposited on the sensor element. According to some embodiments, the property may indicate an effectiveness of the liquid material to react with the substance. In this manner, the liquid material deposited on the sensor element may be examined to ensure that the sensor element is operating with sufficient liquid material and/or sufficiently reactive liquid material. Non-restrictive examples of properties include the following: time, temperature, mass, conductance, transparency, color, fluorescence, refractive index, dielectric coefficient, magnetic coefficient, nonlinear response to stimuli, degradation characteristics, and substance absorption.

The controller may be configured to generate 315 a control signal based on the property. For example, the controller may generate a signal indicating that the liquid material is below a threshold level of effectiveness or that the liquid material is above the threshold level. In an embodiment, the control signal may comprise a continuous signal when the liquid material is below a threshold level of effectiveness that is interrupted when the liquid material is below the threshold level, or vice versa, depending on system requirements.

A liquid dispensing assembly may be arranged 320 to be in communication with the controller. For example, the liquid dispensing assembly may operate to deposit the liquid material on the sensor element. The controller may operate to send control signals to the liquid dispensing assembly indicating whether or not to release the liquid material.

The liquid dispensing assembly may be configured to deposit 325 the liquid material on the sensor element responsive to receiving the control signal. The control signal may take many forms, including one or more discrete (e.g., on/off) signals, a continuous signal, a varying signal, and combinations thereof. For example, the liquid dispensing assembly may release one droplet of liquid material responsive to receiving a discrete control signal from the controller. In another example, the liquid dispensing assembly may continuously release droplets of the liquid material, at specified intervals, while receiving a continuous control signal. In a further example, the control signal may comprise a variable value configured to indicate a liquid dispensing characteristic, such as droplet volume, droplet size, and/or droplet speed. In this manner, the liquid dispensing assembly may release droplets having various characteristics based on the control signal.

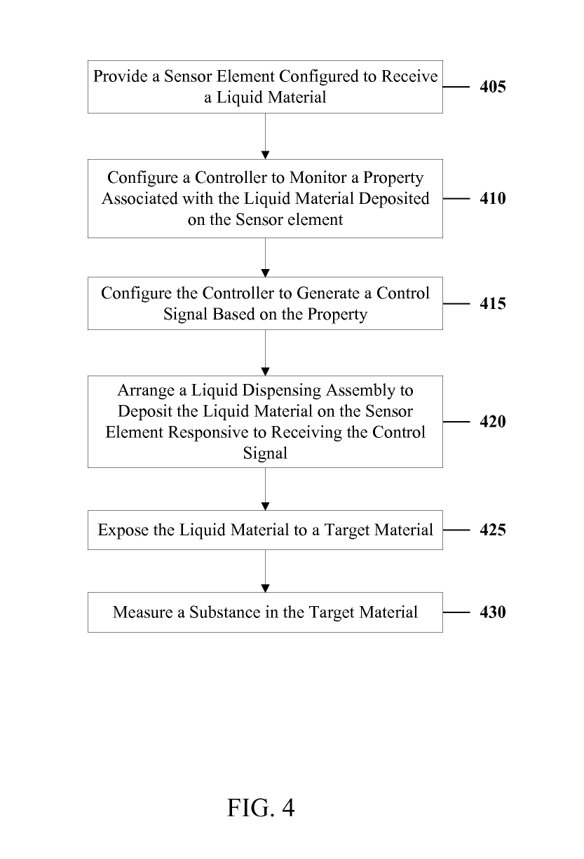

FIG. 4 depicts a flow diagram for an illustrative method of measuring a substance using a sensor element configured according to some embodiments. A sensor element configured to receive a liquid material may be provided 405 for measuring a substance. The sensor element may be comprised of various types of sensors depending on the substance. For example, a SAW sensor may be used to detect chemical analytes, while a metabolism sensor or fluorescent sensor may be used to detect biological analytes.

A controller may be configured to monitor 410 a property associated with the liquid material deposited on the sensor element. In an embodiment, the monitored property may be indicative of the effectiveness of the liquid material to react with the substance, which, in turn, may indicate an ability of the sensor element to detect and/or measure the substance. In an embodiment, the controller may monitor 410 the property directly using one or more sensors or other measuring devices. In another embodiment, the controller may receive information associated with the property from an external source in communication with the controller. For example, the sensor element or other external measuring device may operate to measure a property (e.g., mass, refractive index, temperature, etc.) and to transmit information associated with the property to the controller.

The controller may be configured to generate a control signal based on the property 415. For example, the controller may operate to generate 415 a control signal based on the property information. The control signal may be discrete, continuous, variable, and or combinations thereof. According to some embodiments, the control signal may serve various purposes. For instance, the control signal may signal a cleaning element to clean a sensor element or the control signal may signal a liquid dispensing assembly to start/stop dispensing a liquid material. In an embodiment, the control signal may include information indicating whether the liquid material deposited on the sensor element has degraded. In another embodiment, the control signal may contain information sufficient to control a source of the liquid material.

A liquid dispensing assembly may be arranged to deposit 420 the liquid material responsive to receiving the control signal. In an embodiment, the liquid dispensing assembly may be in communication with the controller and may be configured to deposit 420 the liquid material based on the control signal. For example, the control signal may comprise a discrete signal and the liquid dispensing assembly may release one droplet of liquid material responsive to receiving each discrete signal. In another example, the control signal may comprise a continuous signal and the liquid dispensing assembly may release 420 one droplet of the liquid material at a predetermined interval while receiving the continuous signal.

The liquid material may be exposed 425 to a target material. For example, the liquid material may be deposited on the surface of the sensor element and a liquid and/or gas delivery system may deliver the target material such that the target material contacts 425 the liquid material. In another example, the liquid material may be released from the liquid dispensing assembly such that the liquid material contacts 425 the substance in midair in the space between the liquid dispensing assembly and a surface of the sensor element. In a further embodiment, the liquid material may contact 425 the target material both on the surface of the sensor element and in midair.

A substance in the target material may be measured 430 by the sensor element based on a reaction between the liquid material and a substance of interest in the target material. A measurable reaction may occur when the liquid material contacts the target material. One or more properties of the reaction may be detected by the sensor element. For example, the reaction may cause the temperature of the liquid material, or the target material, to increase. The sensor element may comprise a temperature sensor configured to measure the temperature increase. In an embodiment, the sensor element or a computing device running suitable software may be configured to determine an amount of the substance in the target material based on the measurable reaction.

Some embodiments provide for various forms of measurement. For instance, the mass of the substance may correlate with a temperature increase resulting from a reaction between the liquid material and the substance. In another instance, a sensor element may measure fluorescence resulting from liquid material comprising fluorescent antibodies reacting with target pathogens. For example, a liquid material comprising fluorescent antibodies may be deposited on a substrate such that the liquid material contacts a sample potentially containing a pathogen of interest. The fluorescent antibodies interacting with the target pathogen may emit light when a light (e.g., UV light) is applied from above or from the substrate as in attenuated total reflection (ATR). In another example, the liquid material may comprise an in vivo immunofluorescent solution, for instance, that can detect pathogens and other microbes in water.

Antibodies directed against antigens of the target pathogen can be labeled (e.g., conjugated) with a fluorochrome or fluorescent dye (e.g., fluorescein isothiocyanate or another fluorochrome) for direct immunofluorescence. These fluorescent antibodies may react with the target pathogen, and the preparation may be washed to remove meted fluorescent antibody. The sample is then examined for the target pathogen by ultraviolet light microscopy or another analytical method to detect the immunofluorescent signal (e.g., fluorometry).

Alternatively, secondary fluorochrome-labeled antibodies directed against the primary antibodies (e.g., now serving as antigens) of the species of animal in which the antibodies against the microbe were raised can be used in an indirect immunofluorescence assay. The target microbial antigen is reacted initially with a specific antibody and the resulting antigen-antibody complex is reacted with fluorochrome-labeled antispecies antibody to provide the signal for immunofluorescent detection. In an embodiment, the immunoassay may be deposited on a substrate or sensor element using an inkjet dispensing assembly as described herein. The fluorescence may be monitored over time using a suitable detection technique, such as ATR or other illumination technique. Periodically the assay would be removed using heat decomposition and the process would restart. Additional functionality may be included to ensure the integrity of the analyte detection system, such as an alert activated when the amount of pathogens in a given time frame exceeds a predefined limit.

FIG. 5 depicts an illustrative computing device 500 that may be used to contain or implement program instructions for controlling aspects of an analyte detection system according to some embodiments described herein. In a very basic configuration 502, computing device 500 typically includes one or more processors 504 and a system memory 506. A memory bus 508 may be used for communicating between processor 504 and system memory 506.

Depending on the desired configuration, processor 504 may be of any type including but not limited to a microprocessor (μP), a microcontroller (μC), a digital signal processor (DSP), or any combination thereof. Processor 504 may include one more levels of caching, such as a level one cache 510 and a level two cache 512, a processor core 514, and/or registers 516. The processor core 514 may include an arithmetic logic unit (ALU), a floating point unit (FPU), a digital signal processing core (DSP Core), or any combination thereof. A memory controller 518 may also be used with processor 504. In some implementations the memory controller 518 may be an internal part of processor 504.

Depending on the desired configuration, system memory 506 may be of any type including but not limited to volatile memory (such as RAM), non-volatile memory (such as ROM, flash memory, etc.) or any combination thereof. System memory 506 may include an operating system 520, one or more applications 522, and program data 524. Application 522 may include an analyte detection system manager 526 that is arranged to manage aspects of an analyte detection system in reference to FIGS. 1-4. Program data 524 may include data obtained from operation of the analyte detection system and elements in communication thereto 528. In some embodiments, application 522 may be arranged to operate with program data 524 on operating system 520 such that certain aspects of the analyte detection system, such as a controller and/or sensor element, may operate according to some embodiments described herein. This described basic configuration 502 is illustrated in FIG. 5 by those components within the inner dashed line.

Computing device 500 may have additional features or functionality, and additional interfaces to facilitate communications between basic configuration 502 and any required devices and interfaces. For example, a bus/interface controller 530 may be used to facilitate communications between basic configuration 502 and one or more data storage devices 532 via a storage interface bus 534. Data storage devices 532 may be removable storage devices 536, non-removable storage devices 538, or a combination thereof. Examples of removable storage and non-removable storage devices include magnetic disk devices such as flexible disk drives and hard-disk drives (HDD), optical disk drives such as compact disk (CD) drives or digital versatile disk (DVD) drives, solid state drives (SSD), and tape drives to name a few. Example computer storage media may include volatile and nonvolatile, removable and non-removable media implemented in any method or technology for storage of information, such as computer readable instructions, data structures, program modules, or other data.

System memory 506, removable storage devices 536 and non-removable storage devices 538 are examples of computer storage media. Computer storage media includes, but are not limited to, RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which may be used to store the desired information and which may be accessed by computing device 500. Any such computer storage media may be part of computing device 500.

Computing device 500 may also include an interface bus 540 for facilitating communication from various interface devices (e.g., output devices 542, peripheral interfaces 544, and communication devices 546) to basic configuration 502 via bus/interface controller 530. Example output devices 542 include a graphics processing unit 548 and an audio processing unit 550, which may be configured to communicate to various external devices such as a display or speakers via one or more A/V ports 552. Example peripheral interfaces 544 include a serial interface controller 554 or a parallel interface controller 556, which may be configured to communicate with external devices such as input devices (e.g., keyboard, mouse, pen, voice input device, touch input device, etc.) or other peripheral devices (e.g., printer, scanner, etc.) via one or more I/O ports 558. An example communication device 546 includes a network controller 560, which may be arranged to facilitate communications with one or more other computing devices 562 over a network communication link via one or more communication ports 564.

The network communication link may be one example of a communication media. Communication media may typically be embodied by computer readable instructions, data structures, program modules, or other data in a modulated data signal, such as a carrier wave or other transport mechanism, and may include any information delivery media. A “modulated data signal” may be a signal that has one or more of its characteristics set or changed in such a manner as to encode information in the signal. By way of example, and not limitation, communication media may include wired media such as a wired network or direct-wired connection, and wireless media such as acoustic, radio frequency (RF), microwave, infrared (IR) and other wireless media. The term computer readable media as used herein may include both storage media and communication media.

Computing device 500 may be implemented as a portion of a small-form factor portable (or mobile) electronic device such as a cell phone, a personal data assistant (PDA), a personal media player device, a wireless web-watch device, a personal headset device, an application specific device, or a hybrid device that include any of the above functions. Computing device 500 may also be implemented as a personal computer including both laptop computer and non-laptop computer configurations.

### EXAMPLES

### Example 1

### Sonic Acoustic Wave (SAW) Sensor Element

A SAW device will be used as a sensor element of an analyte detection system and will include interdigitated electrodes arranged on a piezoelectric substrate. Application of a voltage to the electrodes will cause an input transducer of the SAW device to convert the electrical signal to an acoustic wave, which propagates through the substrate to the output transducer, where it will be reconverted into an electric signal. Chemically selective thin film coatings will be applied to the substrate that will shift the frequency of the SAW device. Adsorption of chemicals into the thin films will further shift the propagation velocity and will cause a change in the electrical output of the SAW device. The SAW device will incorporate an electrode to heat the sensor element.

An inkjet dispensing device will deposit the liquid material on the sensor device. The liquid material will be a silicon polymer that will decompose at an elevated temperature, ranging from about 50° C. to about 150° C., and will detect the explosive compound trinitrotoluene (TNT). The inkjet dispensing device will have a first reservoir and a second reservoir. The first reservoir will hold a first liquid material having high sensitivity to TNT. The second reservoir will hold a second liquid material having a low sensitivity to TNT.

The SAW device will be calibrated to the specific mass of the first liquid material. A controller will be in communication with the sensor element of the SAW device and the inkjet dispensing device. The sensor element will be configured to measure the mass of the first liquid material deposited on the surface of the sensor element and to transmit a signal indicating whether the mass of the first liquid material is below a threshold, ranging from about 1 picogram to a about 10 milligrams. The controller will transmit a control signal to the inkjet dispensing device responsive to receiving the signal from the sensor element that the mass of the first liquid material is below a threshold. The control signal will invoke the inkjet dispensing device to deposit a droplet of the first liquid material on the surface of the sensor element.

The inkjet dispensing assembly will dispense the first liquid material on the surface of the sensor element. The SAW device will deliver the target material in liquid form to the sensor element and will continuously measure the resonance frequency. When the correct mass of the first liquid material has been deposited on the sensor element, the sensor element will stop transmitting the signal to the controller that the mass of the first liquid material is below the threshold. The controller will send a control signal to the inkjet dispensing assembly to stop depositing the first liquid material.

The sensor element will detect the presence of TNT. At a time interval determined by the predefined aging behavior of the first liquid material and/or by measurable changes in a shift of the resonance frequency (depending on which occurs first), the sensor element will be heated by the electrode, and the first liquid material will decompose.

The SAW device will communicate to the controller to switch to the second liquid material. The sensor element will be configured to measure the mass of the second liquid material deposited on the surface of the sensor element and to transmit a signal indicating whether the mass of the second liquid material is below a threshold. The controller will transmit a control signal to the inkjet dispensing device responsive to receiving the signal from the sensor element that the mass of the second liquid material is below a threshold. The control signal will invoke the inkjet dispensing device to deposit a droplet of the second liquid material on the surface of the sensor element.

A new liquid material layer including the second liquid material will be deposited. The SAW device will deliver the target material in liquid form to the sensor element and will continuously measure the resonance frequency. The target material will be retested at the lower sensitivity level associated with the second liquid material.

### Example 2

### Measuring Biological Analytes Using Immunofluorescent Liquid Material and Attenuated Total Reflection (ATR)

An analyte detection system will include an inkjet dispensing assembly configured to dispense an immunofluorescent liquid material onto a substrate containing a sample that potentially includes a target pathogen. The inkjet dispensing assembly will be in communication with a controller that includes a timer to identify the amount of time the liquid material has been on the substrate. The substrate will be associated with a heating element that will heat the liquid material to a temperature effective to remove the liquid material from the substrate through heat decomposition.

The liquid material will be generated from antibodies directed against antigens of the target pathogen in solution and labeled with the fluorescent dye fluorescein isothiocyanate. The liquid material will be stored in a reservoir of the inkjet dispensing assembly. The liquid material will be deposited onto the sample on the substrate and the sample-immunofluorescent mixture will be washed to remove unreacted fluorescent antibodies.

The fluorescence resulting from the fluorescent antibodies of the liquid material reacting with the pathogens in the sample may be measured by ATR and monitored over time. After a first specified period of time has expired, as determined by the controller, the controller sends a signal to the inkjet dispensing assembly to discontinue depositing the liquid material. After a second specified period of time has expired, as determined by the controller, the controller will transmit a signal to the heating element. The control signal will activate the heating element and heat the liquid material to a temperature effective to remove the liquid material from the substrate through heat decomposition. When the liquid material has been removed from the substrate, the liquid material deposition process will be repeated, as required.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, compositions or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). While various compositions, methods, and devices are described in terms of “comprising” various components or steps (interpreted as meaning “including, but not limited to”), the compositions, methods, and devices can also “consist essentially of” or “consist of” the various components and steps, and such terminology should be interpreted as defining essentially closed-member groups. It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

Various of the above-disclosed and other features and functions, or alternatives thereof, may be combined into many other different systems or applications. Various presently unforeseen or unanticipated alternatives, modifications, variations or improvements therein may be subsequently made by those skilled in the art, each of which is also intended to be encompassed by the disclosed embodiments.

1. A system for measuring at least one substance using at least one liquid material, the system comprising:
a sensor element comprising a controller operative to monitor at least one property associated with the at least one liquid material deposited on the sensor element, the controller being configured to generate at least one control signal based on the at least one property; and
a liquid dispensing assembly in communication with the controller, the liquid dispensing assembly configured to deposit the at least one liquid material on the sensor element based on the at least one control signal.

2. The system of claim 1, wherein the sensor element comprises at least one of the following: an affinity sensor, an immunosensor, a metabolism sensor, a catalytic sensor, an electrochemical sensor, a fluorescent sensor, a surface acoustic wave sensor, an attenuated total reflection sensor, a piezoelectric sensor, a piezoelectric quartz crystal sensor, a thickness shear mode sensor, a flexural plate wave sensor, an optical absorption sensor, a Fourier-transform infrared (FTIR) optical sensor, a Raman optical sensor, an electrical properties sensor, a magnetic properties sensor, a conductance sensor, a capacitance sensor, a magnetic field orientation sensor, and a bulk acoustic wave resonator sensor.

3. The system of claim 1, wherein the at least one liquid material comprises a high-affinity material configured to measurably react with the at least one substance.

4. The system of claim 1, wherein the at least one liquid material comprises at least one of the following: antibodies, antigens, enzymes, polymers, silicone polymers, carbowax polymers, cyclodextrin polymers, and fluorescent indicators.

5. The system of claim 1, wherein the at least one property is configured to indicate an effectiveness of the at least one liquid material deposited on the sensor element at measuring the at least one substance.

6. The system of claim 1, wherein the at least one property comprises at least one of the following: time, temperature, mass, conductance, transparency, color, fluorescence, refractive index, dielectric coefficient, magnetic coefficient, nonlinear response to stimuli, degradation characteristics, and substance absorption.

7.-8. (canceled)

9. The system of claim 1, wherein the liquid dispensing assembly comprises an inkjet printing assembly.

10.-12. (canceled)

13. A method of preparing a sensor element for measuring at least one substance using at least one liquid material, the method comprising:
providing a sensor element configured to receive the at least one liquid material;
configuring a controller to monitor at least one property associated with the at least one liquid material deposited on the sensor element, the controller being configured to generate at least one control signal based on the at least one property; and
arranging a liquid dispensing assembly in communication with the controller, the liquid dispensing assembly configured to deposit the at least one liquid material on the sensor element responsive to receiving the at least one control signal.

14. The method of claim 13, wherein the sensor element comprises at least one of the following: an affinity sensor, an immunosensor, a metabolism sensor, a catalytic sensor, an electrochemical sensor, a fluorescent sensor, a surface acoustic wave sensor, an attenuated total reflection sensor, a piezoelectric sensor, a piezoelectric quartz crystal sensor, a thickness shear mode sensor, a flexural plate wave sensor, an optical absorption sensor, a Fourier-transform infrared (FTIR) optical sensor, a Raman optical sensor, an electrical properties sensor, a magnetic properties sensor, a conductance sensor, a capacitance sensor, a magnetic field orientation sensor, and a bulk acoustic wave resonator sensor.

15.-16. (canceled)

17. The method of claim 13, wherein the at least one property is configured to indicate an effectiveness of the at least one liquid material deposited on the sensor element at measuring the at least one substance.

18.-24. (canceled)

25. A method of measuring at least one substance in a target material using a sensor element configured to measure the at least one substance using at least one liquid material, the method comprising:
arranging a sensor element to receive the at least one liquid material, the sensor element comprising a controller operative to monitor at least one property associated with the at least one liquid material deposited on the sensor element, the controller being configured to generate at least one control signal based on the at least one property;
arranging a liquid dispensing assembly to deposit the at least one liquid material on the sensor element based on the at least one control signal, wherein the liquid dispensing assembly is in operable communication with the controller; and
exposing the at least one liquid material to the target material such that the sensor element measures for the at least one substance in the target material.

26. The method of claim 25, wherein the at least one liquid material contacts at least a portion of the target material in midair.

27. The method of claim 25, wherein the at least one liquid material contacts at least a portion of the target material on a surface of the sensor element.

28. (canceled)

29. The method of claim 25, wherein the sensor element comprises at least one of the following: an affinity sensor, an immunosensor, a metabolism sensor, a catalytic sensor, an electrochemical sensor, a fluorescent sensor, a surface acoustic wave sensor, an attenuated total reflection sensor, a piezoelectric sensor, a piezoelectric quartz crystal sensor, a thickness shear mode sensor, a flexural plate wave sensor, an optical absorption sensor, a Fourier-transform infrared (FTIR) optical sensor, a Raman optical sensor, an electrical properties sensor, a magnetic properties sensor, a conductance sensor, a capacitance sensor, a magnetic field orientation sensor, and a bulk acoustic wave resonator sensor.

30. The method of claim 25, wherein the at least one liquid material comprises a high-affinity material configured to measurably react with the at least one substance.

31. The method of claim 25, wherein the at least one liquid material comprises at least one of the following: antibodies, antigens, enzymes, polymers, silicone polymers, carbowax polymers, cyclodextrin polymers, and fluorescent indicators.

32. The method of claim 25, wherein the at least one property is configured to indicate an effectiveness of the at least one liquid material deposited on the sensor element at measuring the at least one substance.

33. The method of claim 25, wherein the at least one property comprises at least one of the following: time, temperature, mass, conductance, transparency, color, fluorescence, refractive index, dielectric coefficient, magnetic coefficient, nonlinear response to stimuli, degradation characteristics, and substance absorption.

34.-39. (canceled)

40. A system for measuring at least one substance using at least one liquid material, the system comprising:
a sensor element comprising a controller operative to monitor at least one property associated with the at least one liquid material deposited on the sensor element, the controller being configured to generate at least one control signal based on the at least one property;
a cleaning element operative to clean the sensor element by removing the at least one liquid material deposited on the sensor element based on the at least one control signal; and
a liquid dispensing assembly in communication with the controller, the liquid dispensing assembly configured to deposit the at least one liquid material on the sensor element responsive to the sensor element being cleaned.

41. The system of claim 40, wherein the cleaning element comprises at least one of the following: an ultrasonic element, a thermal element, an electric element, an optical element, and a physical element.

42. The system of claim 40, wherein the sensor element comprises at least one of the following: an affinity sensor, an immunosensor, a metabolism sensor, a catalytic sensor, an electrochemical sensor, a fluorescent sensor, a surface acoustic wave sensor, an attenuated total reflection sensor, a piezoelectric sensor, a piezoelectric quartz crystal sensor, a thickness shear mode sensor, a flexural plate wave sensor, an optical absorption sensor, a Fourier-transform infrared (FTIR) optical sensor, a Raman optical sensor, an electrical properties sensor, a magnetic properties sensor, a conductance sensor, a capacitance sensor, a magnetic field orientation sensor, and a bulk acoustic wave resonator sensor.

43. The system of claim 40, wherein the at least one liquid material comprises a high-affinity material configured to measurably react with the at least one substance.

44. The system of claim 40, wherein the at least one liquid material comprises at least one of the following: antibodies, antigens, enzymes, polymers, silicone polymers, carbowax polymers, cyclodextrin polymers, and fluorescent indicators.

45. The system of claim 40, wherein the at least one property is configured to indicate an effectiveness of the at least one liquid material deposited on the sensor element at measuring the at least one substance.

46. The system of claim 40, wherein the at least one property comprises at least one of the following: time, temperature, mass, conductance, transparency, color, fluorescence, refractive index, dielectric coefficient, magnetic coefficient, nonlinear response to stimuli, degradation characteristics, and substance absorption.

47.-53. (canceled)

54. The system of claim 40, wherein the liquid dispensing assembly comprises:
a plurality of liquid materials, wherein each of the plurality of liquid materials is associated with a sensitivity to the at least one substance,
wherein the cleaning element is operative to clean the sensor element responsive to the sensor element measuring a predetermined amount of the at least one substance, and
wherein the liquid dispensing assembly is configured to deposit one of the plurality of liquid materials associated with a higher sensitivity than a previously deposited liquid material responsive to the cleaning element cleaning the sensor element.

55. The sensor element of claim 40, wherein the liquid dispensing assembly comprises:
a plurality of liquid materials, wherein each of the plurality of liquid materials is associated with an affinity to one of a plurality of substances,
wherein the cleaning element is operative to clean the sensor element responsive to the sensor element measuring a predetermined amount of one of the plurality of substances,
wherein the liquid dispensing assembly is configured to deposit one of the plurality of liquid materials associated with an affinity of another of the plurality of substances responsive to the cleaning element cleaning the sensor element.
